(19) United States
(12) Patent Application Publication
(10) Pub. No.: US 2025/0353012 A1
(43) Pub. Date: Nov. 20, 2025

(54) MICROFLUIDIC ASSAYS AND USES THEREOF

(71) Applicants: National University of Singapore, Singapore (SG); Agency for Science, Technology and Research, Singapore (SG)

(72) Inventors: Matthew Zirui TAY, Singapore (SG); Nicholas Weikang LIN, Singapore (SG); Lih Feng CHEOW, Singapore (SG); Chia-Hung CHEN, Singapore (SG); Laurent Claude Stéphane RÉNIA, Singapore (SG); Lisa Fong Poh NG, Singapore (SG)

(21) Appl. No.: 18/871,044

(22) PCT Filed: May 8, 2023
(86) PCT No.: PCT/SG2023/050317

§ 371 Date: Dec. 2, 2024

(30) Foreign Application Priority Data
May 30, 2022 (SG) 10202250050T

(51) Int. Cl. B01L 3/00; G01N 33/536

(52) U.S. Cl. CPC B01L 3/502784; G01N 33/536; B01L 2200/027; B01L 2200/0605; B01L 2200/0652; B01L 2400/084

(57) ABSTRACT

The present disclosure concerns a microfluidic method of assaying antibody secreting cells (ASCs), comprising the steps of isolating ASCs within droplets such that each droplet encapsulates only one ASC; incubating the droplets of step a) to accumulate antibodies within the droplets; picoinjecting virus into the droplets of step b) to form immune complex droplets; picoinjecting host cells into the immune complex droplets to form neutralised droplets and infected droplets; and sorting the infected droplets from the neutralised droplets, b based on infection of the host cells by the virus, to assay the ASCs within the neutralised droplets. The present disclosure also concerns a microfluidic platform thereof.

### TECHNICAL FIELD

The present invention relates, in general terms, to microfluidic assays and their uses thereof.

### BACKGROUND

Pathogenic viruses are responsible for a multitude of infectious diseases that plagued humanity. As part of our adaptive immune response, neutralizing antibodies (nAbs) play an integral role in conveying protection against viruses. Indeed, in-vivo nAb titers are strongly correlated with protection for a multitude of viral infections, including dengue, SARS-CoV-2 and respiratory syncytial virus (RSV). However, the slow onset of natural nAb production after virus infection (from a few days to weeks) could leave elderly or immunocompromised individuals vulnerable to severe health consequences. In such situations, administration of exogenously produced monoclonal nAbs can often prevent infected individuals from progression to severe stages of disease.

Currently, there are tremendous pressures on nAb discovery efforts to keep up with the rapid mutation of existing virus strains and the emergence of novel viral threats. Although fluorescence-activated sorting (FACS) and display systems can screen a large numbers of antibody-secreting cells (ASCs) for their binding to a particular protein antigen, binding affinity is often not reflective of the true functional efficacy of a nAb candidate. Many high affinity mAbs bind to non-neutralizing epitopes on the viral antigen, rendering them unsuitable for therapeutic applications (FIG. 1a). In the worst case, administration of non-neutralizing Abs could contribute to Antibody-Dependent Enhancement (ADE) effects that increase the severity of multiple viral infections. Meanwhile, cells producing effective nAbs can be directly screened through hybridoma generation and single B cell activation/expansion, but the laborious nature of these procedures results in a long workflow. The majority of the immune repertoire is overlooked due to the low throughput (10²-10³ candidates) nature of existing virus neutralization assays. As such, there is an unmet need for an integrated system that can rapidly perform functional Ab neutralization assay on a large population of ASCs, with the ability to not just identify but also to isolate promising candidates.

Droplet microfluidics platforms present several key advantages that make them ideal for the functional screening of ASCs, which include: 1) high operating throughputs at 10-10⁴ droplets per second, 2) well-established toolkit for droplet manipulation such as merging, splitting and sorting enable complex multi-step assays to be performed, 3) the ability to accommodate a variety of assay reagents type via co-encapsulation, particularly reporter or effector cells needed in most Ab functional assays. While droplet-based ASC screening via Ab binding affinity has been well established, the much more clinically relevant ASC screening via a true virus neutralization assay is still lacking due to the technical challenges of performing the complex multi-step assay. A recent work describes a platform for visualization of virus neutralization by ASCs in microfluidic droplets. Nonetheless, this method is limited to evaluating virus neutralizing activities from 100-1000 droplets in the field-of-view and lacks the critical capability of sorting and retrieving potent nAb secreting cells for downstream analysis or expansion.

It would be desirable to overcome or ameliorate at least one of the above-described problems.

### SUMMARY

To address at least one of the current gaps, the present invention relates to a high-throughput droplet microfluidic system capable of selection and retrieval of ASCs based on the neutralizing function of secreted Abs from single cells. As shown herein as an example, the platform can be used to enrich for cells secreting nAbs against Chikungunya virus (CHIKV). High-throughput screening of functional ASCs with droplet microfluidics can be a new paradigm for the rapid discovery of potent and functional biologics. It is also demonstrated that the present invention can achieve similar enrichment for low frequency (˜2%) functional nAb-producing cells in a background of excess cells secreting irrelevant antibodies, highlighting its potential prospect as a first round enrichment platform for functional ASCs.

The present invention provides a microfluidic method of assaying antibody secreting cells (ASCs), comprising the steps of:
a) isolating ASCs within droplets such that each droplet encapsulates only one ASC;
b) incubating the droplets of step a) to accumulate antibodies within the droplets;
c) picoinjecting virus into the droplets of step b) to form immune complex droplets;
d) picoinjecting host cells into the immune complex droplets to form neutralised droplets and infected droplets; and
e) sorting the infected droplets from the neutralised droplets, based on infection of the host cells by the virus, to assay the ASCs within the neutralised droplets.

In some embodiments, the neutralised droplets are sorted from the infected droplets using a dielectrophoretic sorter.

In some embodiments, the microfluidic method further comprises a step before step a) of generating droplets in the presence of a lipopolysaccharide.

In some embodiments, the microfluidic method further comprises a step after step c) of incubating the immune complex droplets.

In some embodiments, the droplets of step a) and b) are characterised by one or both of:
a volume of about 160 pl to about 200 pl; and
a diameter of about 40 um to about 100 um, or preferably about 70 um to about 90 um.

In some embodiments, the incubation step (step b)) is performed for at least 1 h.

In some embodiments, the microfluidic method further comprises a step after step d) of incubating the neutralised droplets and infected droplets.

In some embodiments, the picoinjection steps are performed using an electric field of about 0.5 Vpp to about 2 Vpp, and with a sinusoidal wave of about 10 kHz to about 30 kHz.

In some embodiments, the method is characterised by a rate of about 200 droplets per second to about 500 droplets per second, or preferably about 300 droplets per second.

In some embodiments, the microfluidic method further comprises a step of recovering the ASCs within the neutralised droplets.

In some embodiments, the recovery step comprises demulsifying the neutralised droplets.

The present invention provides a microfluidic platform, comprising:
a) a droplet generator for generating droplets, each droplet encapsulating one antibody secreting cell (ASC);
b) a first picoinjector chip fluidly connected to the droplet generator, the first picoinjector chip comprising a first nozzle for delivering virus into the droplets to form immune complex droplets;
c) a second picoinjector chip fluidly connected to the first picoinjector chip, the second picoinjector chip comprising a second nozzle for delivering host cells into the immune complex droplets to form neutralised droplets and infected droplets; and
d) a droplet sorter fluidly connected to the second picoinjector chip, for sorting droplets based on infection of the host cells by the virus, the droplet sorter comprising a first channel and a second channel, the second channel configured to have a flow resistance greater than the first channel.

In some embodiments, the second channel is configured to have a flow resistance at least 2 times that of the first channel.

In some embodiments, the droplet generator comprises an aqueous channel for transporting the droplets and 2 oil channels intersecting the aqueous channel, the 2 oil channels configured to flow oil for pinching an aqueous medium in the aqueous channel into droplets.

In some embodiments, the aqueous channel comprises an outlet configured to pinch the aqueous medium into droplets.

In some embodiments, the microfluidic platform further comprises a first vessel fluidly connected to the droplet generator, the first vessel configured to incubate the droplets.

In some embodiments, the microfluidic platform further comprises a second vessel fluidly connected to the first picoinjector, the second vessel configured to incubate the immune complex droplets.

In some embodiments, the microfluidic platform further comprises a third vessel fluidly connected to the second picoinjector, the third vessel configured to incubate the neutralised droplets and infected droplets.

In some embodiments, the microfluidic platform further comprises shielding electrodes.

In some embodiments, the outlet is a constriction.

In some embodiments, the first picoinjector contains fluid containing the virus, the fluid being at a pressure selected to deliver the virus to the droplets.

In some embodiments, the second picoinjector contains fluid containing the virus, the fluid being at a pressure selected to deliver the host cells to the immune complex droplets.

In some embodiments, the first picoinjector is configured with a first electric field, the first nozzle and the first electric field configured to act concurrently to deliver the virus to the droplets.

In some embodiments, the second picoinjector is configured with a second electric field, the second nozzle and the second electric field configured to act concurrently to deliver the host cells to the immune complex droplets.

In some embodiments, the droplet sorter comprises a channel having a width of more than about 100 μm.

In some embodiments, the droplets comprise:
a) a base medium comprising of Dulbecco's Modified Eagle Medium/Nutrient Mixture F-12 (DMEM/F-12), sodium bicarbonate at about 20 mg/L to about 30 mg/L, and sodium pyruvate at about 50 mg/L to about 60 mg/L;
b) fetal bovine serum at about 10% v/v to about 20% v/v of the base medium; and
c) a density gradient medium at about 10% v/v to about 20% v/v of the base medium.

In some embodiments, the droplets further comprise a mixture of penicillin G and streptomycin at about 1% v/v of the base medium.

In some embodiments, a volume of each of the neutralised droplets and infected droplets is about 65 pl to about 1000 pl.

In some embodiments, a volume of the neutralised droplets and infected droplets is less than 2 times a volume of the droplets of step a) and/or b).

In some embodiments, the host cells are delivered at a cell density of about 80 million/mL to about 150 million/mL, or preferably about 100 million/mL.

In some embodiments, the ASCs are B cells or transfected cells, or preferably murine memory B cells.

In some embodiments, wherein the droplets of step b) is characterised by an antibody concentration of about 0.1 μg/mL to about 20 μg/mL, or preferably about 10 μg/mL.

In some embodiments, the volume of each of the immune complex droplets is about 35 pl to about 800 pl.

In some embodiments, the immune complex droplets are characterised by an about 40% to about 60% increase in volume relative to the droplets.

In some embodiments, the immune complex droplets are characterised by a diameter of about 35 μm to about 120 μm, or preferably about 65 μm to about 90 um.

In some embodiments, the immune complex droplets are characterised by a viral titer of about 5 kPFU/μL to about 100 kPFU/μL, or preferably about 75 kPFU/μL.

In some embodiments, the neutralised droplets and infected droplets are characterised by an about 30% to about 40% increase in volume relative to the immune complex droplets.

In some embodiments, the neutralised droplets and infected droplets are characterised by a diameter of about 45 um to about 120 um, or preferably about 70 um to about 100 um.

In some embodiments, the neutralised droplets and infected droplets are characterised by a density of about 5 host cells per droplet to about 15 host cells per droplet.

In some embodiments, the neutralised droplets and infected droplets are characterised by a host cell viability of more than 80% after 30 h of incubation, or preferably more than 90% after 24 h of incubation.

In some embodiments, the recovery step is characterised by an ASC enrichment ratio of more than about 1.8.

In some embodiments, the virus is selected from Chikungunya virus, dengue virus, SARS-CoV-2, respiratory syncytial virus (RSV), Zika virus, EV71, influenza virus, HIV, and norovirus.

### BRIEF DESCRIPTION OF THE DRAWINGS

Embodiments of the present invention will now be described, by way of non-limiting example, with reference to the drawings in which:

FIG. 1 shows a comparison between bulk and single-cell virus neutralization assays. (a) nAbs must bind in a manner to block virus infection. Many Abs selected solely based on their ability to bind to virus lack a neutralizing function. (b) Top: Typical workflow and timeframe required to produce clonal populations of ASCs and screen them for virus neutralizing activity. Bottom: Significant acceleration of functional Ab discovery due to the ability to perform virus neutralization assay in droplets from single cells. Sorting of droplets with high virus neutralizing activity enables retrieval of functional ASCs.

FIG. 2 shows infection of HEK 293T cells with CHIKV-ZSGreen. (a) Bulk infection of HEK 293T cells stained by CHIKV over a period of 44 hours (top and bottom row). Green fluorescence signal increases over time due to replication of CHIKV within the cells (bottom row). (b) Flow cytometry analysis enables enumeration of CHIKV virus infected cells. (c) Degree of infection observed HEK 293T cells were co-incubated with CHIKV virus for 22 hours in the presence of varying amounts of 8B10 neutralizing mAb.

FIG. 3 shows characterization of picoinjectors. (a) 70 μm diameter droplets generated using a standard flow-focusing chip design, (b) droplets after picoinjection of CHIKV to form ≈80 μm diameter droplets and (c) after subsequent cell picoinjection to form ≈90 μm diameter droplets. Scale bar represents 100 μm. (d) Number distribution of picoinjected host HEK 293T cells at various cell densities. (e) In-droplet cell viability over time for droplets picoinjected with 200 million/mL host HEK 293T.

FIG. 4 shows in-droplet CHIKV infection and signal readout. (a) Droplets containing 20 μg/mL of 8B10 nAbs or no nAbs were picoinjected with 37.5 kPFU/μL CHIKV and host HEK 293T cells. Images show droplets at 0 hr and 20 hr from the onset of HEK 293T picoinjection. Scale bar represents 100 μm. (b) Average CHIKV signal intensity of said droplets at the 20 hr time point, quantified via imaging and (c) PMT measurement. (d) The percentage of infected droplets over time at varying picoinjected CHIKV viral titers, as well as in the presence of 8B10 nAbs.

FIG. 5 shows characterization of sorters based on CHIKV infection signal. (a) PMT signal of droplets containing CHIKV infected HEK 293T cells. A sorting pulse is triggered when the PMT signal exceeds the 0.4V threshold in this example. (b) Droplet population containing 50% infected, 50% non-infected droplets before sorting. Image of droplets retrieved at (c) collection and (d) waste outlet after sorting at 0.48V threshold. At least 95% of the infected cells are collected. Scale bar represents 250 μm. (e) ROC curve of sorter obtained by sorting droplets at a range of signal thresholds from 0.12 to 3.24V.

FIG. 6 shows the complete in-droplet single-cell neutralization assay workflow. (a) Single-cell neutralization assay workflow. ASCs (stained red) and mock-transfected cells (stained blue) are first encapsulated in 70 μm diameter droplets and allowed to accumulate nAbs over 24 hrs. Cell staining is merely used for downstream identification of cell types. The droplets were then injected with CHIKV and host HEK 293T cells and incubated to allow infection to take place before they were sorted. (b) Sorted droplets retrieved from the top collection and bottom waste channels respectively when sorted using a 0.4 V threshold. Scale bar represents 100 μm.

FIG. 7 shows droplets retrieved from a single-cell neutralization assay performed using a mixed population of ASCs secreting 8B10 (CHIKV nAbs) and 5A6 (non-relevant SARS-COV-2 nAbs). 8B10 ASCs (stained red) and 5A6 ASCs (stained blue) are subject to the full single neutralization workflow as described in the main text (a) Droplets prior to the sorting process. CHIKV-infected cells exhibit a green fluorescence signal. Sorted droplets retrieved from the (b) top collection and (c) bottom waste channels respectively when sorted using a 0.4 V green fluorescence signal threshold. Scale bar represents 100 μm.

FIG. 8 shows a schematic of a picoinjector chip.

FIG. 9 shows (a) a schematic of a sorter chip. Blue electrodes represent ground electrodes while red electrode represents active electrode. (b) Magnified view of boxed region where sorting occurs.

### DETAILED DESCRIPTION

The present invention is predicated on an understanding that a major bottleneck in antibody development is the search for candidate antibodies with strong functional activity against the desired target. To develop a therapeutic monoclonal antibody, one or several antibody clones with functional activity against the target must be identified and isolated from a vast pool of antibody clones. Current high-throughput methods for such identification either rely on binding to a target-derived antigen, also called a hook (e.g. flow cytometry-based approaches; phage/yeast library approaches), or if not, rely on low-throughput functional assays. The inability to screen candidates at high throughput by function reduces the chance to find the best antibody candidates from the vast available pool of clones. Recently, methods have been developed that utilize microfluidic devices to generate picoliter to nanoliter-sized water-in-oil droplets in a fluorinated oil medium. However, most of these platforms perform selection using Ab binding affinity against an antigen as a proxy for functional efficacy instead of using function-based assays. Using a droplet-based functional assays to enrich for functionally relevant ASCs is inherently difficult, due to the increased biological complexity of the assay compared to simple binding assays, which imposes technical limitations that have thus far not been compatible with the high throughput required for antibody screening at a scale comparable to traditional antibody discovery technologies. Firstly, the sequential addition of reagents, commonly done by droplet-to-droplet merging, is unable to achieve 100% efficiency of merging due to the high dependency on the synchronization of the two droplets. This lowers the specificity of the assay at high throughput, which is a critical parameter for identification of rare antibody clones. Secondly, function-based assays require the ability to keep cells alive and functional within droplets for long durations, which requires a large droplet volume for nutrition and waste buffering. However, a large droplet volume results in a slower sorting speed due to its larger mass.

Additionally, other microfluidic assay designs used 1-to-1 droplet matching, which is limited in being more dependent on synchronicity of the paired droplets and thus a higher likelihood of failure in the delivery of virus/cell cargo to the original droplet. Other microfluidic assay designs have used droplets of a smaller size (<70 um), which do not supply sufficient nutrition for robust mammalian cell culture.

The present invention provides a microfluidic method of assaying antibody secreting cells (ASCs), comprising the steps of:
a) isolating ASCs within droplets such that only one ASC is encapsulated within one droplet;
b) incubating the droplets of step a) in order to accumulate antibodies within the droplets;
c) picoinjecting virus into the droplets of step b) in order to form immune complex droplets;
d) picoinjecting host cells into the immune complex droplets in order to form neutralised droplets and infected droplets; and
e) sorting the neutralised droplets from the infected droplets in order to assay the ASCs within the neutralised droplets.

The volume of each of the neutralised droplets and infected droplets can be about 300 pl to about 400 pl. The volume of the neutralised droplets and infected droplets can be less than 2 times the volume of the initial droplets of step a).

The present assay uses picoinjection to enable high-throughput delivery of virus and reporter cells/host cells, which more robustly delivers cargo into all droplets, necessary for a high-specificity neutralisation assay. Additionally, this assay instead can produce droplets of an optimal size which are large enough for culturing mammalian cells for the duration of the bioassays, while remaining small enough for efficient droplet sorting. The assay also includes a sorting step to enrich for cells secreting nAbs, demonstrating its potential application for the discovery of mAbs against viral diseases.

The microfluidic assay as disclosed herein is capable of screening at least 100,000 candidates, and ideally >1 million candidates. In comparison, traditional antibody discovery techniques can only screen up to around ˜10,000 candidates with manual operation, and ˜100,000 candidates with robotic operations.

The inventors have also found that other factors can be relevant: Time: The time taken to run all the droplets through the microfluidic chip is preferably less than 2 hours, due to loss of cell viability over time at room temperature, and introduction of additional variation in droplet nutritional status and infection time that increase the assay variability. This imposes lower limits on the necessary droplet throughput on each of the steps to at least 140 Hz, assuming 1:10 candidate:droplet ratio (10% droplet occupancy, necessary given Poisson limitations to achieve single cell per droplet). For example, at a 5% single-cell occupancy rate, the screening time is about 2.8 h; at a 10% single-cell occupancy rate, the screening time is about 1.4 h; at a 20% single-cell occupancy rate, the screening time is about 0.7 h.

Droplet merging throughput: Standard droplet merging does not achieve the throughput required, typically ranging from 10-100 droplets/second. In contrast, the present process of picoinjection can achieve the throughput of, for example, about 300 droplets/second, which may achieve a processing throughput of 1 million droplets/hr.

Droplet sorting throughput: The droplet merging process and also nutritional requirements for conducting the bioassay used in the paper result in very large droplet volumes (˜5 nl droplets). At these volumes, standard droplet sorting does not achieve the throughput required, and in fact droplets of such a size cannot be sorted with current sorting designs since sufficiently large dielectrophoretic forces cannot be achieved without breaking up the droplets due to the increased inertia associated with droplets of larger mass. In contrast, the picoinjection procedure allows reliable delivery of small volumes of fluid to each droplet, allowing us to have a small final droplet size after all reagent addition (200 pl). Together with an optimized sorting design conveying sufficiently large dielectrophoretic forces, while preventing undesired coalescence of droplets via the addition of extra electric field shielding elements, we are capable of achieving the sorting throughput required of about 200 droplets/s.

Reliability: Droplet merging requires precise control of the periodicity of the droplets, and in this regard, it is easy for desynchronization to occur, especially as the droplet frequency increases (e.g. to increase throughput). This will lead to high rates of false positives when Ab-secreting cells are not properly co-encapsulated with host cells or infectious agents. In contrast, picoinjection can be made very reliable at adding the reagent into all droplets passing through, and if there are malfunctions in the process (e.g. pressure too high), the main effect is the formation of additional small droplets that would not contain the antibody-secreting cells, and therefore do not affect sensitivity or specificity.

In some embodiments, the droplets have a ASCs occupancy rate of about 6% to about 15%, or about 6% to about 20%. The occupancy rate refers to the volume taken up by entities such as cells in the droplet relative to the total volume of the droplet. In other embodiments, the occupancy rate is about 7% to about 15%, about 8% to about 15%, about 9% to about 15%, about 10% to about 15%, about 10% to about 14%, about 10% to about 13%, or about 10% to about 12%. In other embodiments, the occupancy rate is about 10%.

In some embodiments, the microfluidic method further comprises a step before step a) of generating droplets in the presence of a lipopolysaccharide. For example, lipopolysaccharides from Escherichia coli 0111:B4 may be used.

In some embodiments, the droplets of step a) and b) are characterised by a volume of about 30 pl to about 600 pl. In other embodiments, the volume is about 30 pl to about 550 pl, about 30 pl to about 500 pl, about 30 pl to about 450 pl, about 30 pl to about 400 pl, about 30 pl to about 350 pl, about 30 pl to about 300 pl, about 30 pl to about 250 pl, about 30 pl to about 200 pl, about 30 pl to about 150 pl, about 30 pl to about 100 pl, about 30 pl to about 80 pl, about 30 pl to about 70 pl, about 30 pl to about 60 pl, about 30 pl to about 50 pl, about 50 pl to about 600 pl, about 100 pl to about 600 pl, about 150 pl to about 600 pl, about 200 pl to about 600 pl, about 250 pl to about 600 pl, about 300 pl to about 600 pl, about 350 pl to about 600 pl, about 400 pl to about 600 pl, about 450 pl to about 600 pl, or about 500 pl to about 600 pl. Preferably, the droplets can be characterised by a volume of about 160 pl to about 200 pl.

In some embodiments, the droplets of step a) and b) are characterised by a diameter of about 40 μm to about 100 μm, or preferably about 70 μm to about 90 um.

In some embodiments, the ASCs is B cells. In other embodiments, the ASCs is murine memory B cells, including VDJ-humanized murine memory B cells, or human memory B cells. In other embodiments, the ASCs are cells that have been transfected or transduced to secrete antibody. In other embodiments, the ASCs is transfected cells. Transfection commonly refers to the introduction of nucleic acids into eukaryotic cells, or more specifically, into animal cells. Classically, the term transfection was used to denote the uptake of viral nucleic acid from a prokaryote-infecting virus or bacteriophage, resulting in an infection and the production of mature immune complex particles. However, the term has acquired its present meaning to include any artificial introduction of foreign nucleic acid into a cell. In transfection, the introduced nucleic acid may exist in the cells transiently, such that it is only expressed for a limited period of time and does not replicate. As such, transiently transfected genetic material is not passed from generation to generation during cell division, and it can be lost by environmental factors or diluted out during cell division. However, the high copy number of the transfected genetic material leads to high levels of expressed protein within the period that it exists in the cell. Alternatively, it may be stable and integrate into the genome of the recipient (transduction), replicating when the host genome replicates.

Being able to use and assay B cells (for example) makes it possible to circumvent the long procedure of generating hybridoma cells and facilitate the screening of a much larger fraction of the immune repertoire, including that of humans and animals with humanized antibody systems.

The cell culture media required for supporting co-culture of all biological agents necessary for neutralization can also be optimized, resulting in good assay quality leading to sufficient specificity and sensitivity to allow use in enrichment and/or isolation of hits.

In some embodiments, the droplets comprise:
a) a base medium comprising of Dulbecco's Modified Eagle Medium/Nutrient Mixture F-12 (DMEM/F-12), sodium bicarbonate at about 20 mg/L to about 30 mg/L, and sodium pyruvate at about 50 mg/L to about 60 mg/L;
b) fetal bovine serum at about 10% v/v to about 20% v/v of the base medium; and
c) a density gradient medium at about 10% v/v to about 20% v/v of the base medium.

It was found that the above medium is optimal in general, especially given the need for co-culture of different cell types in this microfluidic system.

For example, sodium bicarbonate can be at about 24.5 mg/L and sodium pyruvate at about 55 mg/L. The density gradient medium at a concentration of about 15% v/v. The density medium can be Optiprep.

In some embodiments, the droplets further comprise a mixture of penicillin G and streptomycin at about 1% v/v of the base medium.

The incubation step (step b)) can be performed in an incubation chamber or vessel. The vessel can be removed from the chip. With off-chip incubation, as the droplets need to incubate for long periods of time at various steps of the procedure (e.g. for antibody accumulation, immune complex formation, and infection), parameters such as temperature and oxygen level can be more easily regulated. As the droplets may be coated with a surfactant, they do not coalesce.

In some embodiments, the incubation step (step b)) is performed for at least 1 h, 2 h, 4 h, 8 h, 10 h, 12 h, 14 h, 16 h, 18 h, 20 h, 22 h, 24 h, 36 h, or 48 h. In some embodiments, the incubation step (step b)) is performed at a temperature of about 30° C. to about 50° C.

The droplets may proceed to the next step once sufficient antibodies have accumulated within the droplets. In some embodiments, the droplets of step b) is characterised by an antibody concentration of about 0.1 μg/mL to about 20 μg/mL. In other embodiments, the concentration is about 0.2 μg/mL to about 20 μg/mL, about 0.4 μg/mL to about 20 μg/mL, about 0.5 μg/mL to about 20 μg/mL, about 0.7 μg/mL to about 20 μg/mL, about 0.8 μg/mL to about 20 μg/mL, about 1 μg/mL to about 20 μg/mL, about 2 μg/mL to about 20 μg/mL, about 4 μg/mL to about 20 μg/mL, about 6 μg/mL to about 20 μg/mL, about 8 μg/mL to about 20 μg/mL, about 8 μg/mL to about 18 μg/mL, about 8 μg/mL to about 16 μg/mL, about 8 μg/mL to about 14 μg/mL, about 8 μg/mL to about 12 μg/mL, or about 8 μg/mL to about 10 μg/mL. In other embodiments, the concentration is about 10 μg/mL.

The average antibody concentrations in the droplets can vary depending on the type of cells, antibody potency and assay detection limit. For example, if the antibody has a high potency, a lower average antibody concentration can be used, thus reducing the incubation period.

After droplet formation, the virus is picoinjected into the droplets to form immune complex droplets. Picoinjection is a controlled way to add reagents to droplets. It is insensitive to variations in the periodicity of the drops, allowing uniform injection even if the drops enter irregularly. The volume of reagent added may be adjusted by either varying the time of the injection, or the injection pressure. Picoinjection may be triggered using an electric field which destabilise the surfactants on the droplets. To stop picoinjecting, the electric field need only be switched off, allowing the surfactants already present on the droplets to re-stabilize the interface.

It was found that picoinjection can avoid excessive droplet size that would otherwise occur by merging three droplets together (ACS, virus and host cell), which can reduce throughput and reliability. Picoinjection can also improve the accuracy of the assay. In particular, by maintaining the volume of the droplets to a suitable size, the duration for the accumulation of antibodies, virus-antibody interaction and host cells infection can be reduced, thereby improving the throughput.

In some embodiments, the picoinjection step is performed using an electric field of about 0.5 Vpp to about 2 Vpp, and with a sinusoidal wave of about 10 kHz to about 30 kHz. The sinusoidal wave can be amplified 100-fold. In other embodiments, electric field is about 1 Vpp, and a sinusoidal wave of about 20 kHz.

In some embodiments, the virus is picoinjected at a volume of about 20 μL to about 120 μL. In other embodiments, the volume is about 25 μL to about 120 μL, about 30 μL to about 120 μL, about 35 μL to about 120 μL, about 40 μL to about 120 μL, about 40 μL to about 110 μL, about 40 μL to about 100 μL, about 40 μL to about 95 μL, about 40 μL to about 90 μL, about 40 μL to about 85 μL, or about 40 μL to about 80 μL.

In some embodiments, the volume of each of the immune complex droplets is about 35 pl to about 800 pl, about 40 pl to about 800 pl, or about 40 pl to about 750 pl. In other embodiments, the volume of each of the immune complex droplets is about 35 pl to about 700 pl, about 35 pl to about 650 pl, about 35 pl to about 600 pl, about 35 pl to about 500 pl, about 35 pl to about 400 pl, about 35 pl to about 300 pl, about 40 pl to about 300 pl, about 50 pl to about 300 pl, about 60 pl to about 300 pl, about 70 pl to about 300 pl, about 80 pl to about 300 pl, about 90 pl to about 300 pl, about 100 pl to about 300 pl, about 120 pl to about 300 pl, or about 150 pl to about 300 pl. Preferably, in some embodiments, the volume of each of the immune complex droplets is about 165 pl to about 250 pl.

In some embodiments, the volume of the immune complex droplets is less than 1.8 times a volume of the droplets, less than 1.7, less than 1.6, less than 1.5, or less than 1.4.

In some embodiments, the immune complex droplets are characterised by an about 40% to about 60% increase in volume relative to the droplets of step a) and/or b). In other embodiments, the volume increase is about 45% to about 60%, about 50% to about 60%, or about 50% to about 55%.

In some embodiments, the immune complex droplets are characterised by a diameter of about 35 um to about 120 um, about 35 um to about 110 um, about 35 um to about 100 um, about 35 um to about 90 um, about 35 um to about 80 um, about 35 um to about 70 um, about 35 um to about 60 um, about 40 um to about 50 um; about 95 um to about 120 um, or about 100 um to about 110 um. In some embodiments, the immune complex droplets are characterised by a diameter of about 65 um to about 90 um, or preferably about 75 um to about 85 um.

The concentration of the virus may be controlled such that all the viruses may be complexed within the immune complex droplet if the appropriate antibody is present.

In some embodiments, the immune complex droplets are characterised by a viral titer of about 5 kPFU/μL to about 100 kPFU/μL, about 10 kPFU/μL to about 100 kPFU/μL, about 15 kPFU/μL to about 100 kPFU/μL, about 20 kPFU/μL to about 100 kPFU/μL, about 25 kPFU/μL to about 100 kPFU/μL, about 30 kPFU/μL to about 100 kPFU/μL, about 40 kPFU/μL to about 100 kPFU/μL, about 50 kPFU/μL to about 100 kPFU/μL, about 60 kPFU/μL to about 100 kPFU/μL, about 70 kPFU/μL to about 100 kPFU/μL, about 70 kPFU/μL to about 90 kPFU/μL, about 70 kPFU/μL to about 80 kPFU/μL, or preferably about 75 kPFU/μL. A viral titer of about 5 kPFU/μL corresponds to an average of 1 virus per 200 pl droplet. In some embodiments, the viral titer is at least about 12 kPFU/μL.

In some embodiments, the microfluidic method further comprises a step after step c) of incubating the immune complex droplets. The incubation allows the viruses to fully complex with the antibodies.

In some embodiments, the incubation step is performed for at least 5 minutes, 10 minutes, 15 minutes, 30 minutes, 1 h, 2 h, or 4 h.

In some embodiments, the incubation step is performed in a vessel.

As used herein, “neutralised droplets” refer to droplets comprising ASC, antibodies, virus and host cells, and which a substantial amount of host cells are not infected. For example, as a positive control, when 20 μg/mL 8B10 (mouse antibody) is added, only 9.75% of cells are infected, and thus exhibit lower level of infection; i.e. neutralised. Accordingly, when less than 20% of the cells are infected in the droplet, the droplet can be characterised as a neutralised droplet. Corollary, “infected droplets” refer to droplets in which a substantial amount of host cells are infected; i.e. more than 20%.

In some embodiments, the host cells are delivered to the immune complex droplets at a rate of about 200 droplets per second to about 500 droplets per second, about 250 droplets per second to about 500 droplets per second, about 300 droplets per second to about 500 droplets per second, about 300 droplets per second to about 450 droplets per second, about 300 droplets per second to about 400 droplets per second, or preferably about 300 droplets per second.

In some embodiments, the host cells are delivered at a cell density of about 80 million/mL to about 150 million/mL, about 80 million/mL to about 140 million/mL, about 80 million/mL to about 130 million/mL, about 80 million/mL to about 120 million/mL, about 80 million/mL to about 110 million/mL, about 90 million/mL to about 110 million/mL, or preferably about 100 million/mL.

In some embodiments, the host cells is picoinjected at a volume of about 20 pL to about 120 pL. In other embodiments, the volume is about 25 pL to about 120 pL, about 30 pL to about 120 pL, about 35 pL to about 120 pL, about 40 pL to about 120 pL, about 45 pL to about 120 pL, about 50 pL to about 120 pL, about 50 pL to about 115 pL, or about 50 pL to about 110 pL.

In some embodiments, the volume of each of the neutralised droplets and infected droplets is about 65 pl to about 1000 pl, about 100 pl to about 1000 pl, or about 200 pl to about 1000 pl. In other embodiments, the volume of each of the neutralised droplets and infected droplets is about 65 pl to about 85 pl, about 90 pl to about 115 pl, about 115 pl to about 140, about 700 pl to about 800 pl, about 750 pl to about 900 pl, or about 800 pl to about 1000 pl. In some embodiments, the volume of each of the neutralised droplets and infected droplets is about 250 pl to about 320 pl, about 270 pl to about 380 pl, or about 300 pl to about 400 pl.

In some embodiments, the volume of the neutralised droplets and infected droplets is less than 1.9 times a volume of the droplets, less than 1.8, less than 1.7, less than 1.6, or less than 1.5.

In some embodiments, the neutralised droplets and infected droplets are characterised by an about 30% to about 40% increase in volume relative to the immune complex droplets.

In some embodiments, the neutralised droplets and infected droplets are characterised by a diameter of about 45 um to about 120 um, or about 70 um to about 120 um. In other embodiments neutralised droplets and infected droplets are characterised by a diameter of about 45 um to about 65 um, or preferably about 50 um to about 60 um; about 100 um to about 125 um, or preferably about 110 um to about 120 um. In some embodiments, the neutralised droplets and infected droplets are characterised by a diameter of about 70 um to about 100 um, or preferably about 80 um to about 90 um.

In some embodiments, the neutralised droplets and infected droplets are characterised by a density of about 5 host cells per droplet to about 15 host cells per droplet, about 7 host cells per droplet to about 15 host cells per droplet, or about 7 host cells per droplet to about 10 host cells per droplet.

In some embodiments, the droplets have a ASCs and host cell occupancy rate of about 6% to about 15%. In other embodiments, the occupancy rate is about 7% to about 15%, about 8% to about 15%, about 9% to about 15%, about 10% to about 15%, about 10% to about 14%, about 10% to about 13%, or about 10% to about 12%. In other embodiments, the occupancy rate is about 10%.

In some embodiments, the microfluidic method further comprises a step after step d) of incubating the neutralised droplets and infected droplets.

In some embodiments, the incubation step is performed for at least 12 h, 14 h, 16 h, 18 h, 20 h, 22 h, 24 h, 36 h, or 48 h.

In some embodiments, the incubation step is performed in a vessel.

In some embodiments, the neutralised droplets and infected droplets are characterised by a host cell viability of more than 80% after 30 h of incubation, or preferably more than 90% after 24 h of incubation.

In both the picoinjection steps to form the immune complex droplets and the neutralised droplets and infected droplets, a fluidic resistor (such as a narrow serpentine channel) may be added to the viral solution and host cell solution in order to stabilise the injection process.

The assay also comprises a sorting step which provide sufficient piezoelectric force for sorting large droplets, allowing high-throughput sorting of the larger droplet sizes used in this assay.

In some embodiments, the neutralised droplets are sorted from the infected droplets via a dielectrophoretic sorter. The sorter can be subjected to an electric force of about 100 Hz to about 200 Hz, about 100 Hz to about 180 Hz, about 100 Hz to about 160 Hz, about 100 Hz to about 150 Hz, about 110 Hz to about 150 Hz, about 120 Hz to about 150 Hz, about 130 Hz to about 150 Hz, or about 140 Hz to about 150 Hz.

The sorting step is capable of enriching for neutralised droplets even when they are present at low frequencies. The enrichment was not negatively impacted by the presence of large number of cells secreting non-relevant antibodies.

In some embodiments, the microfluidic method further comprises a step of recovering the ASCs within the neutralised droplets. In some embodiments, the recovery step comprises demulsifying the neutralised droplets. The mixture of oil and aqueous medium can then be separate to isolate the aqueous medium (for example via centrifugation), from which the ASCs can be recovered.

In some embodiments, the recovery step is characterised by an ASC enrichment ratio of about 1.8 to about 3, or preferably about 1.90 to about 2.75. In other embodiments, the ASC enrichment ratio is more than about 1.8, about 2, about 2.2, about 2.4, about 2.6, about 2.8 or about 3.

In some embodiments, the virus is selected from Chikungunya virus, dengue virus, SARS-CoV-2, respiratory syncytial virus (RSV), Zika virus, EV71, influenza virus, HIV, and norovirus.

In some embodiments, the method is characterised by a rate of about 200 droplets per second to about 500 droplets per second, or preferably about 300 droplets per second.

In some embodiments, the method is characterised by a throughput of about 100 droplets/s (or Hz) to about 400 droplets/s, or preferably about 200 droplets/s to about 300 droplets/s. In some embodiments, each step is independently characterised by a throughput of about 100 droplets/s to about 400 droplets/s, or preferably about 140 droplets/s to about 300 droplets/s.

In some embodiments, the method (from step a to e) is characterised by a duration of less than 2 h.

The present invention also provides a microfluidic platform, comprising:
a) a droplet generator for generating droplets, each droplet encapsulating one antibody secreting cell (ASC);
b) a first picoinjector chip fluidly connected to the droplet generator, the first picoinjector chip comprising a first nozzle for delivering virus into the droplets to form immune complex droplets;
c) a second picoinjector chip fluidly connected to the first picoinjector chip, the second picoinjector chip comprising a second nozzle for delivering host cells into the immune complex droplets to form neutralised droplets and infected droplets; and
d) a droplet sorter fluidly connected to the second picoinjector chip, for sorting droplets based on infection of the host cells by the virus, the droplet sorter comprising a first channel and a second channel, the second channel configured to have a flow resistance greater than the first channel.

In some embodiments, the microfluidic platform comprises:
a) a droplet generator comprising at least one aqueous channel having a width of about 40 μm to about 100 μm, the aqueous channel for transporting droplets, each droplet encapsulating one antibody secreting cell (ASC);
b) a first picoinjector chip fluidly connected to the droplet generator, the first picoinjector chip comprising a first nozzle and a first electric field, the first nozzle and the first electric field configured to act concurrently to deliver virus into the droplets in order to form immune complex droplets;
c) a second picoinjector chip fluidly connected to the first picoinjector chip, the second picoinjector chip comprising a second nozzle and a second electric field, the second nozzle and the second electric field configured to act concurrently to deliver host cells into the immune complex droplets in order to form neutralised droplets and infected droplets; and
d) a droplet sorter fluidly connected to the second picoinjector chip, the droplet sorter comprising a top channel and a bottom channel, the bottom channel configured to have a flow resistance at least 2 times that of the top channel in order to sort the neutralised droplets and infected droplets.

The volume of each of the neutralised droplets and infected droplets can be about 300 pl to about 400 pl. The volume of the neutralised droplets and infected droplets can be less than 2 times a volume of the droplets.

In some embodiments, the droplet generator further comprises 2 oil channels intersecting the aqueous channel, the 2 oil channels configured to flow oil for pinching an aqueous medium in the aqueous channel into droplets.

In some embodiments, the aqueous channel comprises an outlet configured to pinch the aqueous medium into droplets. In some embodiments, the outlet is a constriction.

In some embodiments, the microfluidic platform further comprises a first vessel fluidly connected to the droplet generator, the first vessel configured to incubate the droplets.

In some embodiments, the microfluidic platform further comprises a second vessel fluidly connected to the first picoinjector, the second vessel configured to incubate the immune complex droplets.

In some embodiments, the microfluidic platform further comprises a third vessel fluidly connected to the second picoinjector, the third vessel configured to incubate the neutralised droplets and infected droplets.

In some embodiments, the microfluidic platform further comprises shielding electrodes.

In some embodiments, the first picoinjector contains fluid containing the virus, the fluid being at a pressure selected to deliver the virus to the droplets. In some embodiments, the first picoinjector is configured with a first electric field, the first nozzle and the first electric field configured to act concurrently to deliver the virus to the droplets.

In some embodiments, the second picoinjector contains fluid containing the host cells, the fluid being at a pressure selected to deliver the host cells to the immune complex droplets. In some embodiments, the second picoinjector is configured with a second electric field, the second nozzle and the second electric field configured to act concurrently to deliver the host cells to the immune complex droplets.

In some embodiments, the droplet sorter comprises a channel having a width of more than about 100 μm. Accordingly, each channel may be more than about 100 μm wide.

### Working Principles of the Microfluidics Platform

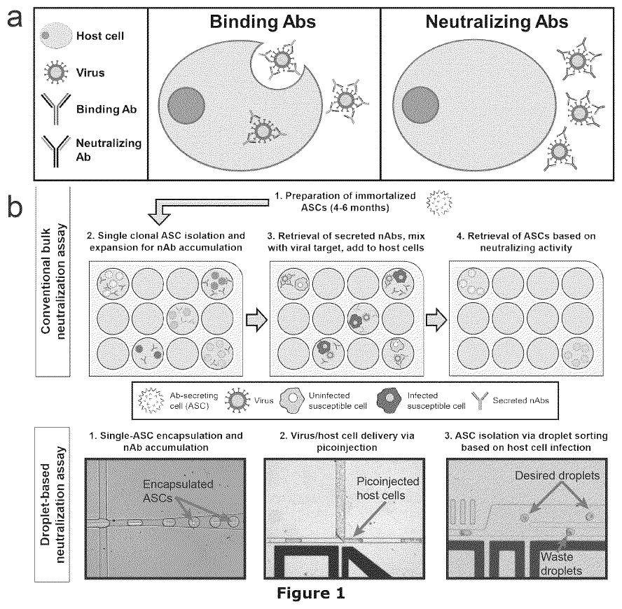

The conventional workflow for screening and isolation of ASCs with virus neutralization capability is shown in FIG. 1b (top). Due to the limited proliferation capability of patient-derived Ab-secreting B cells, they must first be immortalized or stimulated to proliferate (e.g. via hybridoma formation or cytokine addition)—a process that is both time-consuming and low-yield. Following this, single B cells are isolated in well plates (e.g. 96 or 384 well plates) and clonally expanded. This expansion step, which could take many weeks to months, is necessary to obtain sufficient Ab concentrations within the large volumes (10-100 μL) of well plates. Subsequently, secreted Abs are recovered from each well, mixed with a pre-determined amount of target virus particles for binding to occur, and finally incubated with susceptible host cells to assess the degree of virus infection. A very important final step is the specific retrieval of ASCs based on their neutralizing activities for downstream sequencing or expansion. This conventional workflow is inadequate in meeting the needs of rapid nAb discovery against novel viruses and their variants as it requires months of processing, and is limited to screening a limited number of ASCs.

It is reasoned that the most time-consuming step of the current workflow (i.e. cell immortalization and clonal expansion) can be eliminated if secreted Abs from a single ASC can approach concentration levels needed for effective virus neutralization. Confinement of single ASC in small volumes would allow rapid accumulation of Abs as diffusion is prevented. Accordingly, in this platform, single ASCs are encapsulated within picoliter droplets to facilitate rapid Ab accumulation (FIG. 1b, bottom). This is followed by delivery of live virus to each droplet to allow for binding, and finally delivery of susceptible host cells into the same droplets for virus infection to occur. Most importantly, the present platform allows high throughput interrogation (at a rate of 700,000 droplets/hour) of virus infection and integrated sorting and recovery of droplets with low virus infection where neutralization has occurred.

A detailed description of the workings of the invention is laid out below. In the embodiments that follows, the invention is described in relation to some conditions for consistency to showcase the present invention. However, the skilled person would understand that the invention is not limited to such.

As an example, a Chikungunya virus (CHIKV) infection model was used to validate the principles and performance of the platform. Chikungunya virus is a re-emerging pathogen that is endemic in Africa and many parts of Asia, with massive outbreaks with case numbers in the millions in recent decades. There are currently no clinically licensed vaccines or treatments available for Chikungunya infection, but a monoclonal nAb treatment has shown positive results in a Phase I clinical trial. The ability to rapidly screen for monoclonal nAbs is pertinent to the development of better therapy regimens for Chikungunya infections.

### 1.1 Bulk Infection and Neutralization Assay

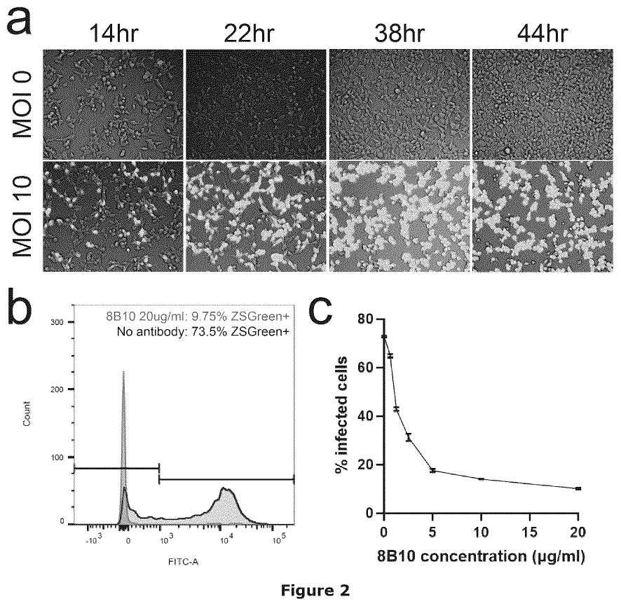

We first characterized the bulk infection behaviour of CHIKV. The CHIKV-ZSGreen strain encodes a fluorescent ZSGreen protein under the control of a subgenomic promoter. Infection and replication of CHIKV-ZSGreen in host cell would produce a green fluorescence signal. As expected, CHIKV-ZSGreen infection of HEK 293T cells in 96 well plates resulted in an increase of cells expressing green fluorescence over time, plateauing at 22 hours with 73% of infected cells at a multiplicity of infection (MOI) of 10. Additionally, to investigate the infection behavior of CHIKV in the presence of nAbs, a previously reported anti-CHIKV monoclonal nAb, 8B10 is allowed to bind to CHIKV before susceptible cells are added. Addition of 8B10 nAb resulted in dose-dependent reduction of infection in HEK 293T cells (FIG. 2b). When 20 g/mL 8B10 is added, only 9.75% of cells are infected (FIG. 2c).

1.2 A Staged Approach to Performing an in-Droplet Virus Neutralization Assay

A conventional bulk virus neutralization assay typically comprises of four steps: 1. Single-cell isolation of ASCs, followed by clonal expansion for Ab accumulation over time in the supernatant. 2. Mixing of Ab-containing supernatant and virus to allow binding to occur. 3. Addition of Ab-virus mixture onto host cells for infection to occur. 4. Measurement of virus infection and retrieval of ASC candidates that have virus neutralization activities. Similarly, to adapt such an assay into a droplet-based setting, multiple microfluidic operations are required as follows; 1. Single-cell isolation of ASCs (without requiring clonal expansion) via droplet encapsulation and accumulation of Abs in droplets. 2. Virus delivery into droplets via picoinjection. 3. Host cells delivery into droplets via picoinjection. 4. Measurement of in-droplet virus neutralization and ASC isolation via droplet sorting.

### 1.2.1 ASC Encapsulation

ASCs were first singly encapsulated into 70 μm diameter droplets using a standard flow-focusing chip, then incubated over a period of 24 hours for nAb accumulation. The average concentration of nAbs in each ASC-containing droplet at 24 hours was quantified via ELISA to be approximately 10 μg/mL, which is well within the nAb concentration required to produce effective virus neutralization in bulk (FIG. 2c).

### 1.2.2 Virus and Host Cell Delivery Via Picoinjection

The workflow is a multi-step process requiring the sequential addition of viruses and host cells. Thus, implementing a robust method to perform these operations is crucial in ensure the quality of the final assay. Previous reports of an in-droplet virus neutralization assay relied on droplet-to-droplet merging system to combine ASCs, virus and host cells, with a final droplet volume of approximately 5 nL. However, droplet-to-droplet merging is highly dependent on the synchronicity of the pair of droplets, and any disruption to this (e.g. arising from a few coalesced droplets) can result in poor merging efficiency. Moreover, while large droplets sizes are acceptable for visualization, they are incompatible with high-throughput droplet sorting due to the tendency of larger droplets to break when sorted at high speeds if ASC retrieval is desired.

A strategy was developed using picoinjection to deliver virus and host cells into ASC-containing droplets. Since picoinjection delivers only a fraction of the incoming droplet's volume, contents can be delivered successfully without significantly increasing droplet size. The self-triggering mechanism of picoinjection also results in robust performance even when droplet periodicity may change over the course of the experiment. This ensures that reagents are delivered to all droplets to maximise the proportion of droplets where CHIKV infection occurs. After each round of picoinjection, the droplet diameters were observed to increase by approximately 10 μm, or a corresponding volume increase of 50.9% and 35.8% respectively.

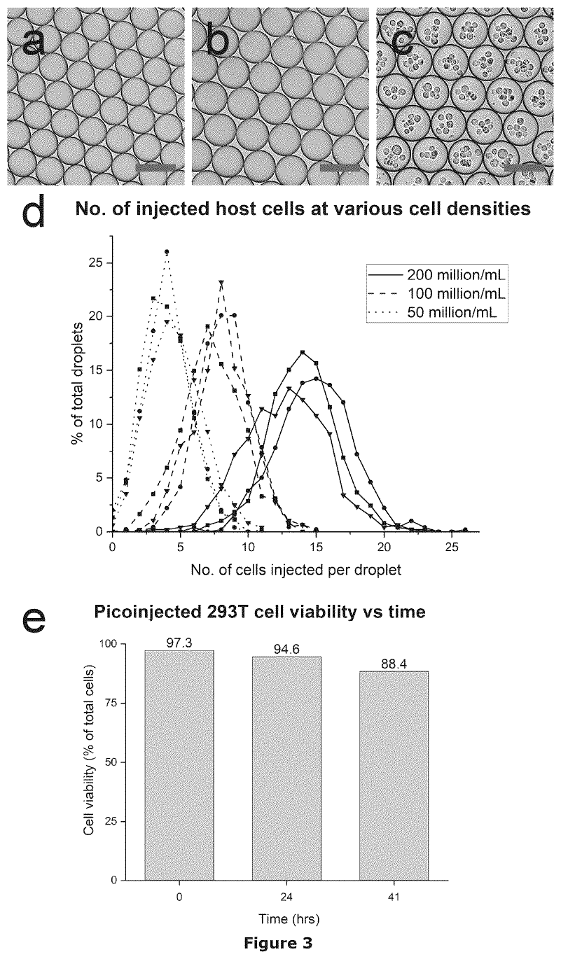

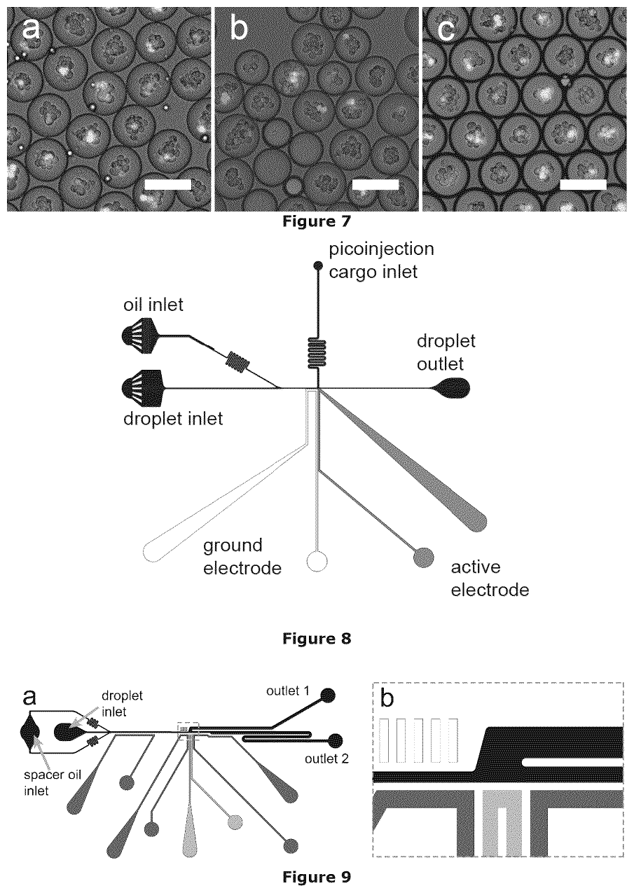

An exemplary schematic of a picoinjector chip is shown in FIG. 8. In the workflow, a picoinjector chip is first used to deliver CHIKV into fully-formed 70 μm diameter droplets containing single ASC. Subsequently, a second picoinjector chip delivers host cells into droplets at a rate of 300 droplets per second. High cell densities ranging from 50 to 200 million cells/mL were used to ensure that multiple host cells can be delivered to each droplet. FIG. 3d showed that the number of host cells delivered into each droplet can be controlled by varying the cell densities. Finally, we investigated the viability of host cells that were delivered via picoinjection. Cell viability remained high at 94.6% at 24 hours (FIG. 3e), indicating that the process was suitable for in-droplet infection to occur. Likewise, the excellent viability of multiple cells (88.4%) over 41 hours of incubation in droplets clearly shows that sufficient nutrients are available to sustain both the ASCs and host cells throughout the workflow. The much-enhanced viability of cells in droplet could in part be attributed to the optimized nutrient-rich culture media in droplets and improved gas-exchange of droplets during incubation in 12-well plates (Methods).

1.3 in-Droplet Infection and Purified nAb Neutralization

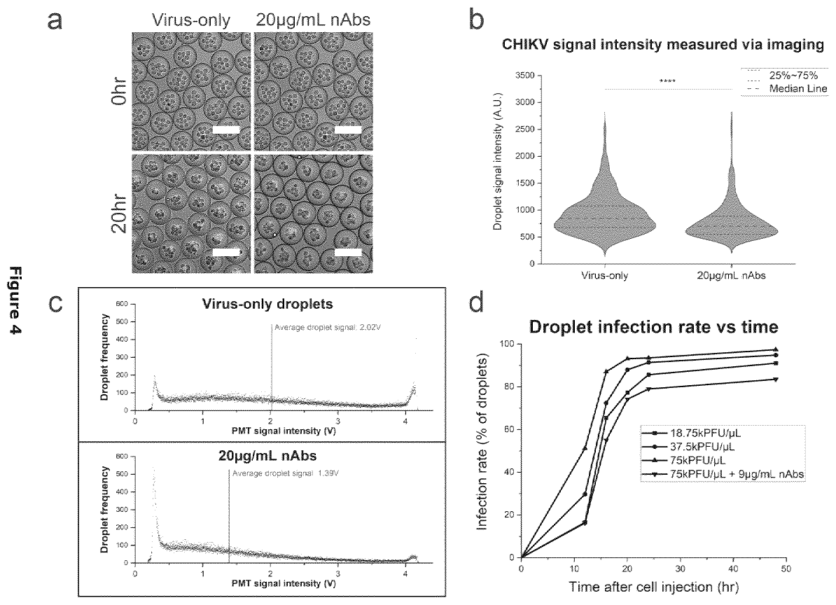

In-droplet CHIKV infection was performed by adding 37.5 kPFU/μL CHIKV into 70 μm diameter droplets containing cell culture media, followed by the addition of host cells by picoinjection. For all in-droplet infection experiments, an average of 8 host cells were delivered to each droplet during picoinjection (100 million cells/mL at injection port). Twenty hours after host cells addition, detectable green fluorescence signals are observed in a majority of droplets, indicating successful in-droplet virus infection (FIG. 4a).

To investigate the effect of nAbs on CHIKV infection, droplets containing 20 μg/mL of purified 8810 nAbs were injected with 37.5 kPFU/μL of CHIKV, and in-droplet infection was compared against a virus-only control (FIG. 4a). The droplet signal intensities (average of 2D image) were analysed via imaging (FIG. 4b) and compared against a continuous-flow PMT detection system (peak fluorescence of each droplet) (FIG. 4c). A significant reduction in the average CHIKV infection signal was observed in the majority of the nAb containing droplets.

To determine the optimal viral titer to be used in the subsequent single-cell neutralization experiments, two conditions need to be considered. Firstly, the CHIKV viral titer to be used should achieve a sufficiently high infection rate needed to reduce the number of false positives during the downstream droplet sorting step. Secondly, to reduce the probability of false negatives which stem from infection even in the presence of nAbs, the viral titer should be kept as low as possible. As such, there is a delicate balance between the infection duration and viral titer to define a window of specificity. The infection rate of droplets was studied for different viral titers over a period of 48 hours after host cell injection (FIG. 4d). In general, the higher the picoinjected viral titer, the greater the percentage of infected droplets at all time points. Additionally, the infection rate appeared to plateau after 24 hours regardless of viral titer used. Based on the results, a viral titer of 75 kPFU/μL was selected to be used for the single-cell neutralization assay as it provided a high droplet infection rate at 93.2% beyond 20 hours. As a positive control for the presence of nAbs, droplets containing 9 μg/mL of 8B10 nAbs (to simulate the average concentrations of Abs accumulated in droplets after 24 hours) are injected with 75 kPFU/μL CHIKV virus, followed by host cell injection for infection. We observed that the percentage of droplets with infected cells is consistently lower at all times when droplets contain nAbs, demonstrating their protective effects against CHIKV infection in this droplet-based assay. In this way, collection of droplets containing cells exhibiting a lower level of infection is expected to enrich for nAbs.

### 1.4 Droplet Sorter Characterization

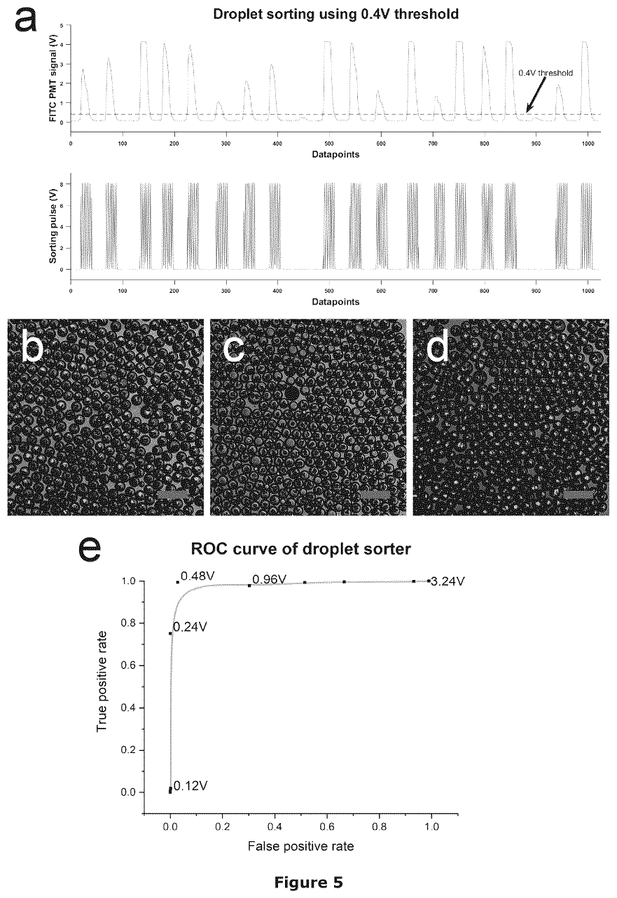

The previously reported droplet-based viral neutralization assay does not allow downstream retrieval of ASCs secreting nAbs as it lacks the key enabling step for isolation of nAb-containing droplets. To address this gap, the workflow integrates a 100 μm height droplet dielectrophoretic sorters that is capable of isolating droplets based on the CHIKV infection signal (FIG. 9). To characterize the performance of this sorter, a mixed pool containing 1:1 ratio of droplets with CHIKV-infected host cells and droplets with uninfected host cells were sorted based on the detected CHIKV fluorescence signal. The high fluorescence of droplets containing infected cells triggers a series of sorting pulses to redirect them into a waste reservoir (FIG. 5a). We defined a positive droplet as one that contains uninfected cell (an indication of presence of nAb). The false positive and true positive rates of the sorted droplets were then assessed over a range of sorting thresholds ranging from 0.12 to 3.24V (FIG. 5e). The images of the droplets before sorting (FIG. 5b), at the collection reservoir (FIG. 5c) and in the waste reservoir (FIG. 5d) at a sorting threshold of 0.48V demonstrates the highly accurate performance of the integrated sorter in the microfluidic platform.

1.5 Application of the Microfluidic Platform to Enrich for Cells Secreting nAbs Against CHIKV Infection

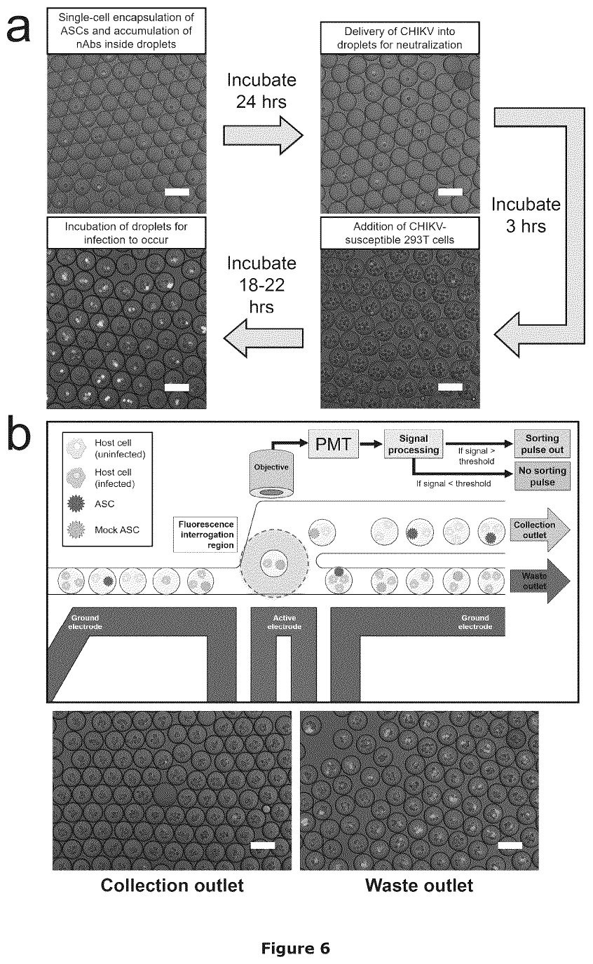

Finally, we validate the performance of the platform for recovery of cells secreting nAbs against CHIKV infection. We prepared an initial population of ASCs that consists of a mixture of cells that secrete 8B10 nAbs (stained red) and cells that do not secrete nAbs (stained blue) at a 1:2 ratio. Note that the cell staining is not used for cell sorting, but rather for the downstream identification of cell types. Single cells from this mixed population are encapsulation in 70 μm diameter droplets and incubated for 24 hours to allow nAb accumulation in the droplets. Subsequently, CHIKV virus and host cells are sequentially added into the droplets and allowed to incubate for 18-22 hours for infection to occur (FIG. 6a). Droplet sorting was then performed using sorting thresholds of 0.4V and 0.6V, in accordance to the ROC curve established previously (FIG. 6b). The sorted droplets were then demulsified and the recovered cells were analyzed using flow cytometry to determine the percentages of ASCs (stained red) and non-secreting cells (stained blue) (Table 1). While flow cytometry showed that the percentage of nAb secreting cells is 26-33% in the initial population, in experiments done over three separate occasions, the percentage of nAb secreting cells is higher in the collected set of droplets (42-57%). To reflect the efficiency of enriching nAb secreting cells, we adopted the “enrichment ratio” measure, defined as the percentage ratio of ASC to non-secreting cells after and before sorting. An ASC enrichment ratio of 1.90-2.75 was obtained using the platform, in a proof of concept for selecting ASCs secreting nAb against CHIKV.

The relatively modest enrichment ratio of virus neutralization assay compared to antibody affinity assays is well within expectations. Unlike antigen-binding assay involves simple molecular interactions between antigen and antibody, virus neutralization assay is much more complicated involving the binding of antibody to the right epitope, and requires complex cellular mechanisms that determines virus infectivity and host cell permissiveness. Incomplete permissiveness of the host cells (also seen in bulk neutralization assay, where ˜30% of cells are uninfected even when 25 there is no neutralizing Ab (FIG. 2C)) could result in false positive droplets being selected, thereby reducing the enrichment ratio. Nevertheless, this is the first demonstration of high-throughput virus neutralizing antibody enrichment in a semiautomatic microfluidics platform, and could already be used as a first enrichment step to provide a coarse selection of nAb secreting cells. We believe that this demonstration sets the foundation to its future use for a variety of viral diseases.

TABLE 1
Distribution of ASCs and mock transfected cells
before and after sorting using a 0.4 V and 0.6
V threshold for 3 independent experimental runs.

| | Pre-sorting | | Post-sorting | | | |
|---|---|---|---|---|---|---|
| | | % of mock | Sorting | | % of mock | Enrich- |
| Rep. | % of | transfected | threshold | % of | transfected | ment |
| no. | ASCs | cells | (V) | ASCs | cells | ratio |
| 1 | 33.33 | 66.67 | 0.4 | 56.98 | 43.02 | 2.65 |
| | | | 0.6 | 57.87 | 42.13 | 2.75 |
| 2 | 26.67 | 73.33 | 0.4 | 46.67 | 53.33 | 2.41 |
| | | | 0.6 | 42.14 | 57.86 | 2.00 |
| 3 | 33.08 | 66.92 | 0.4 | 51.36 | 48.64 | 2.14 |
| | | | 0.6 | 48.43 | 51.57 | 1.90 |

Further, in addition of the results of ASC enrichment from non-secreting cells using the present invention, similar enrichment of specific neutralizing antibody secreting cells can be achieved in more complex scenarios that better resembles physiological samples. Two important aspects were focused upon: 1) The proportion of neutralizing antibody secreting cells are low (0.1-2%) in convalescent/immunized patients, 2) There is an excess of B cells secreting unrelated antibodies in patient samples. In order to simulate these conditions, an experiment was conducted where two kinds of cells secreting different monoclonal antibodies were prepared (8B10 is CHIKV neutralizing antibody, and 5A6 is a non-relevant SARS-COV-2 neutralizing antibody). ASCs secreting 8B10 (labelled red) and 5A6 (labelled blue) were mixed at a 1:50 ratio and the sorting experiment was performed as above. Representative images of the droplets before sorting, as well as sorted droplets from the top collection and bottom waste channel are shown in FIG. 7a-c. As before, the unsorted droplets consist of mostly virus infected cells and a mixture of specific (red) and non-relevant (blue) antibody secreting cells. Droplets recovered from the top collection channel contains predominantly uninfected cells, and an observable enrichment of specific CHIKV neutralizing antibodies (red). Upon FACS analysis of the recovered cells, we observed that the sorted cells contains 6.27% of CHIKV neutralizing antibody secreting cells, up from 2.69% in the unsorted population (Table 2). This enrichment ratio of 2.42 at sorting threshold of 0.4 V is similar to what we obtained in the earlier experiment. The results showed that the present invention retain the ability to enrich for neutralizing antibody secreting cells even when they are present at low frequencies, and that the enrichment was not negatively impacted by the presence of large number of cells secreting non-relevant antibodies. The demonstration of successful application of the present invention in a scenario that better simulates physiological condition is an important milestone towards clinical implementation of this platform.

TABLE 2
Distribution of CHIKV-specific ASCs (8B10) and ASCs secreting irrelevant
antibodies (5A6) before and after sorting on the present platform.

| Pre-sorting | | Post-sorting | | | |
|---|---|---|---|---|---|
| % of | % of | Sorting | % of | % of | |
| specific | irrelevant | threshold | specific | irrelevant | Enrichment |
| ASC (8B10) | ASC (5A6) | (V) | ASC (8B10) | ASC (5A6) | ratio |
| 2.69 | 97.31 | 0.4 | 6.27 | 93.73 | 2.42 |
| | | 0.6 | 4.39 | 95.61 | 1.66 |

### DISCUSSION

The emergence of novel viral threats and rapid mutation of virus strains have posed considerable challenges for conventional screening methods to rapidly discover effective nAbs for treating infected individuals. Herein, a rapid (<3 days), high throughput (150,000 cells per hour) integrated microfluidic platform capable of detecting and sorting ASC based on their virus neutralization activity is shown. Sorted live ASCs could be amenable to downstream expansion for large-scale production of nAb, or characterized with techniques such as single cell RNA sequencing to identify Ab-encoding gene. This platform could be a new paradigm for significantly shortening the workflow for virus neutralizing Ab discovery and production.

This is the first time an in-droplet virus neutralization assay has been demonstrated in the context of an infectious human disease where there is clinical evidence for the therapeutic benefits of exogenously administered nAb. Previous reports on in-droplet virus neutralization assay are only limited to characterizing the functional activities of ASCs, as it lack droplet sorting ability. Here, we successfully developed and optimized the microfluidic platform with functional ASC retrieval as an end goal, in order to realize its true potential for accelerating high throughput discovery of virus nAbs. Cells secreting monoclonal antibodies are used to validate the present platform to statistically evaluate the in-droplet virus neutralizing activities. As virus neutralizing activity is performed at the single antibody-secreting cell level, it is compatible with rapid method to generating antibody-secreting cells (e.g. transient transfection) that does not require lengthy cell immortalization. On the other hand, there is also great potential to apply this method for enrichment of functional population from polyclonal antibody secreting cells, such as what would be typically found from convalescent patients.

In this example, we demonstrate that a single round of workflow enriches functional ASCs relative to non-Ab-secreting cells by 1.90-2.75 fold. Similar enrichment of specific ASC was also achieved when they were at a low proportion (˜2%) within large excess of cells secreting a irrelevant antibody, indicating a promising prospect of applying this method for enriching functional antibodies from primary samples. The relatively modest enrichment factor is consistent with a partially permissive host cell that we have used in this proof of concept. Various literature have shown the choice of host cells and reporter virus could have a significant impact on the sensitivities of virus neutralization assay. We believe there are significant opportunities for host cell and reporter virus engineering that could improve their permissiveness and infectivity for droplet based virus neutralization assay. For example, knocking out antiviral genes in host cells (e.g. STAT1) could drastically improve virus infection rates, as shown in Dengue Virus. Overexpression of viral receptors in host cells (e.g. ACE2 and TMPRSS2 for SARS-CoV-2, MXRA8 for CHIKV) could also significantly increase the efficiency of virus entry into host cells. Engineering approaches to improve virus capsid stability and improved virus manufacturing process to reduce empty capsids would also increase the infection rates of virus. These approaches would be expected to significantly increase the specificity of the platform for selection of virus-neutralizing ASCs by reducing the false positives.

Notwithstanding the additional specificities that could be gained from host cells and reporter virus engineering, the current enrichment of functional ASCs from convalescent individuals or immunized animals already represents a significant advance on yielding a subpopulation of ASCs secreting more potent polyclonal nAbs for immediate applications. We note that despite our best efforts to follow the conventional virus neutralizing assay steps in the single cell droplet workflow, there are unavoidable differences. Among them, the variabilities between the number of target cells, as well as heterogeneity among the antibody-secreting cells and virus, will be accentuated when they are present in small numbers within a droplet. As such, we envision the present invention as the first enrichment step to provide a coarse selection of neutralizing antibody producing cells. If desired, this enriched population could be used to generate polyclonal antibodies with better functionalities compared to the unsorted populations. A second round of workflow on the collected cells can also be performed to improve on the enrichment of ASCs. Furthermore, we envision that the workflow can be performed with ASCs enriched from previously reported Ab binding affinity assays, in order to obtain nAbs with high affinity. Finally, RNA sequencing of the antibody-coding genes can be performed on cells that are enriched using workflow. Antibody sequences that are enriched relative to the initial population can be identified. Such approaches have been very successfully used to identify therapeutic antibodies even when the specific ASCs constitute a small fraction of the circulating cells (e.g. from convalescence patient). A combination of enrichment through this workflow and RNA sequencing is expected to enable rapid identification of functional antibodies from a polyclonal mixture.

In summary, we present a complete platform for the rapid discovery and retrieval of functional ASCs. We demonstrated an example of the workflow to enrich for cells secreting nAbs against the CHIKV virus. In reality, the platform is versatile for rapid screening of biologics for treatment of various viral infections. We envision that it will be of great interest to the scientific community seeking to characterize nAbs against particular viruses, and biotechnology companies interested in adopting a new paradigm for rapid functional biologics discovery.

### Methods

### Device Fabrication and Operation

The polydimethylsiloxane (PDMS) microfluidic chips used in this study were made using well-established soft lithography fabrication techniques. Three types of microfluidic chips—droplet generator, picoinjector, droplet sorter—were used to establish the in-droplet viral neutralization assay. Flow control of both aqueous and oil phases in all processes was performed by syringe pumps (Pump 11 Elite, Harvard Apparatus, Holliston, MA) running at infusion mode. Oil phase used in the study is made up of fluorocarbon oil Novec™ HFE-7500 (3M, Singapore) containing 1% (w/w) Picosurf-1™ surfactant (Sphere Fluidics, Cambridge, UK) to stabilize droplets.

A typical 60 μm height, 3-inlet flow-focusing channel design was used for droplet generation and single-cell encapsulation processes. The aqueous and oil channel widths were designed to be at 50 μm, which then constricted to 40 μm at the outlet to facilitate droplet breakup. 70 μm diameter droplets were generated by infusion of aqueous and oil. Next, to deliver CHIKV and host cells into droplets, 45 μm height/width picoinjectors with a 40 μm picoinjector nozzle width were used. To generate the electric field required to disrupt the stable interface of reinjected droplets for picoinjection to occur, a 1 Vpp 20 kHz sinusoidal wave was amplified 100-fold and passed into the electrodes of the picoinjector. Lastly, to sort droplets after CHIKV infection, 100 μm height droplet sorters were used. To ensure that droplets preferentially enter the top outlet channel in the absence of an electric field, the bottom outlet channel was lengthened such that its resistance is approximately 2 times the top channel. Additional shielding electrodes were included around the device to prevent undesired coalescence of droplets from the sorting electric pulses.

To fabricate the master moulds needed to create the microfluidic devices, SU-8 2050 negative photoresist (Kayaku Advanced Materials, Westborough, MA) was first spin coated onto silicon wafers. Subsequent UV exposure via a mask aligner (MJB4, SUSS MicroTec, Germany) and development was performed according to the SU-8 product sheet's recommended settings. The retrieved moulds were then surface-treated with trichloro-(1H,1H,2H,2H-perfluorooctyl)silane (Sigma-Aldrich, St. Louis, MO) in a dessicator overnight. PDMS (Sylgard 184TM, Dow Corning Inc, Midland, MI) was then added over the moulds, degassed, and cured. The cured PDMS microchannels are then removed from the moulds using a scalpel, followed by creation of inlets and outlets using a 1 mm biopsy punch. The microchannels were then cleaned by ultrasonication for 10 minutes, dried, and irreversibly bonded to a substrate using a plasma cleaner (PDC-32G, Harrick Plasma, Ithaca, NY). Glass slides which were spin-coated with PDMS were used as the substrate for droplet generators, whereas uncoated glass slides were used as the substrate for picoinjectors and sorters. After plasma bonding, to create the electrodes required for the picoinjectors and sorters, a low-melting point indium alloy wire (WIREBN-52189, Indium Corporation, Clinton, NY) was melted into the electrode inlet channels, then connected to wires and secured with UV-curable glue (Uni-Seal™ 6322, Incure, Asheville, SC).

### CHIKV-ZSGreen Virus Production

CHIKV (LR2006 OPY1)-ZsGreen infectious molecular clones were used to make viral stocks, which were then passaged up to two times in Vero E6 cells to amplify viruses. Virus stocks were concentrated by ultracentrifuging at 28,000 g for 4 hours with a sucrose cushion. Viral stock titres were quantified via plaque assay with serial dilution on Vero E6 cells.

### Bulk Infection and Neutralization Assay

For infection assays in bulk conditions, HEK 293T cells were plated in 96-well plates at 30,000 cells per well, and incubated for 5 hours to allow cell adhesion. Meanwhile, antibody stocks were mixed with CHIKV-ZSgreen virus stocks (6,000 pfu/μl) for 2 hrs at 37° C. to allow immune complexes to form. 100 μl of virus+antibody mix were then added to cells, and incubated at 5% C02, 37° C. for 22 hours. Cells were then trypsinized and fixed for readout by flow cytometry. Flow cytometry was performed on a BD LSR II flow cytometer.

### Generation of Monoclonal Antibody-Secreting Cells

A single plasmid (encoding for 8B10 or 5A6) was constructed to express both the heavy and light chain genes of the specific antibody with the human ferritin heavy and light chain promoters respectively. HEK 293T cells were plated in a 6-well plate overnight, then transfected at around 80% confluency with the 8B10 plasmid using Lipofectamine 3000, following the manufacturer's protocol. Cells were harvested via trypsinization 24 hours post-transfection for use in assays.

in-Droplet CHIKV Infection and Purified nAb Neutralization Assay

In-droplet infection assays were performed at three different CHIKV viral titers (18.75/37.5/75 kPFU/μL) to identify the optimal titer to be used in subsequent neutralization experiments. 70 μm diameter droplets were first generated using two aqueous phases—the first comprised growth media only (DMEM/F12 containing 20% FBS and 1% Penstrep) while the second comprised growth media containing 15.5% (v/v) Optiprep (Sigma-Aldrich, St. Louis, MO). The droplets were collected into syringes and incubated for 24 hours in an incubator with 5% CO₂ at 37° C. The droplets were then picoinjected with CHIKV, collected into another syringe and returned into the incubator for 3 hours before being subjected to a round of host cell picoinjection. For the host cell picoinjection process, host cells were resuspended with 15.5% (v/v) Optiprep to achieve a cell density of 100 million/mL and loaded into a syringe containing a magnetic stir bar. During the picoinjection process, the syringe was kept on ice and the stir bar is continuously agitated to ensure homogeneity of the exiting cell suspension. After the second round of picoinjection, the droplets were transferred into a 12-well plate to improve gas exchange (Corning® Costar® TC treated 12-well flat-bottom plate, Sigma-Aldrich, St. Louis, MO) containing 2 mL of HFE-7500 added with 1% (wt/wt) Picosurf-1TM, sealed with parafilm and left to incubate in incubator with 5% C02 at 37° C. At different time points, the droplets were sampled and loaded into a disposable hemocytometer (EVE™ Cell counting slides, NanoEntek, Seoul), followed by imaging via fluorescence microscopy. Infection rates were then quantified based on the CHIKV green fluorescence signal using ImageJ. Infected droplets were defined as droplets containing at least one host cell with a fluorescence intensity exceeding the background.

In the purified nAb in-droplet neutralization experiment, purified 8B10 nAbs diluted with growth media to obtain a concentration of 18 μg/mL was used as the first aqueous phase. After dilution with the secondary aqueous phase, a final in-droplet nAb concentration of 9 μg/mL was obtained. The droplets were then subjected to the above mentioned in-droplet infection workflow to determine the infection rate of nAb-containing droplets over time.

### Optical and Electronics Setup for Droplet Sorting

The optical setup used in droplet sorting comprised of a fluorescence light source (SPECTRA III, Lumencor Inc., USA) and a photomultiplier tube (PMT) detection system (H9306-03, Hamamatsu Photonics K.K., Japan). Only the FITC channel was used in all droplet sorting experiments.

Voltage signals obtained from the PMT were parallelized into two outputs for sorting and signal recording. For signal recording, the PMT analog signals were converted to digital signals using a data acquisition card (USB-6002, National Instruments, USA) and recorded using the in-built Analog Input Recorder application in MATLAB (R2019b, MathWorks, USA). For sorting, PMT signals were processed in real-time using an Arduino DUE microprocessor (Arduino, USA) to determine if a particular droplet signal exceeds a pre-defined threshold. The DUE microprocessor was used to control an Arduino UNO microprocessor (Arduino, USA) responsible for generating 8 Vpp, 10 kHz square waves which were amplified 100-fold through a high-voltage amplifier before they were passed into the sorter's electrodes. Upon detection of a PMT signal which exceeds the threshold, sorting waves were switched on for 1750 μs to actuate the droplet towards the bottom sorting channel.

For ROC curve characterization, a 50:50 mixed pool of infected and non-infected droplets were sorted at 11 different thresholds ranging from 0.12 to 3.24V. 0.5 μL of droplets from each sorting condition was then sampled and fluorescently imaged to identify the number of true and false positives.

Table 3 shows the PMT signal distribution of droplets containing 293T cells without virus.

TABLE 3
PMT signal distribution

| | Average | | 95 | 99 | 99.2 | 99.5 |
|---|---|---|---|---|---|---|
| | droplet | | percen- | percen- | percen- | percen- |
| | signal | Median | tile | tile | tile | tile |
| Signal | 0.240 | 0.229 | 0.386 | 0.470 | 0.480 | 0.502 |
| intensity | | | | | | |
| (V) | | | | | | |

### Single-Cell Droplet Neutralization Assay

A single-cell in-droplet neutralization assay was performed to verify the ability of the workflow in enriching for ASCs secreting functional nAbs against CHIKV. A cell suspension comprising a mix of CHIKV nAb-secreting ASCs and non-secreting cells in a ratio of 1:2 was first resuspended in 15.5% (v/v) Optiprep. For later identification between the two populations of cells, the ASCs were stained red (CellTracker™ Red CMTPX Dye, Invitrogen, Waltham, MA) while the non-secreting cells were stained blue (Cell Proliferation Dye eFluor™450, Invitrogen, Waltham, MA) beforehand. Seventy μm diameter droplets were then generated using the cell suspension as the first aqueous phase and growth media as the secondary aqueous phase. A 20% single-cell encapsulation rate was used. The droplets were collected into a syringe, placed in an incubator with 5% C02 at 37° C. for 24 hours to allow nAb accumulation within the droplets, before they were subjected to the in-droplet infection assay workflow. Droplet sorting was performed between 18-22 hours after host cell picoinjection when an overall droplet infection rate of >90% was achieved. The resulting droplets from each sorting condition and the pre-sorting baseline were then fluorescently imaged, demulsified using 20% (v/v) 1H,1H,2H,2H-Perfluoro-1-octanol (Sigma-Aldrich, St. Louis, MO) in HFE7500, and the aqueous phases were recovered. Cells retrieved from the droplets were trypsinized and fixed, then analysed by flow cytometry for percentages of ASCs and non-secreting cells.

To evaluate the performance of the present invention for enrichment of specific functional ASCs in the presence of large excess of cells secreting irrelevant antibodies, 8B10 ASCs (secreting CHIKV nAbs) and 5A6 ASCs (secreting non-relevant SARS-COV-2 nAbs) are subject to the workflow as described above. An initial cell population comprising of 8B10 and 5A6 ASCs at a ratio of 1:50 was first encapsulated in 70 μm diameter droplets to achieve a single-cell occupancy rate of 20%. The droplets were then incubated to allow the accumulation of Abs within the droplets over a period of 24 hrs. The droplets were then picoinjected with 75kPFU/uL of CHIKV, incubated for 3 hrs to allow Ab neutralization of CHIKV, before a second picoinjection step to deliver 293T host cells at a cell density of 100 million/mL. The droplets were then incubated for 20 hrs to allow infection to take place before they were dielectrophoretically sorted at a 0.4/0.6V signal threshold.

### Droplet Media Composition

Table 4 shows the average concentration of nAbs recovered from the supernatant of droplets containing ASCs. 70 μm diameter droplets containing 10% singly-encapsulated ASCs were incubated for 24 hours to allow accumulation of nAbs prior to demulsification and quantification via ELISA. DMEM/F12+20% FBS (highlighted blue) was used for all in-droplet processes stated in the main text as it exhibited the best performance.

TABLE 4
Average concentration of nAbs in droplet

| | Average | | Average Ab |
|---|---|---|---|
| | droplet Ab | Average droplet | concentration of |
| | concentration | Ab concentration | cell-containing |
| | at 0 hour | at 24 hour | droplets at 24 |
| Droplet media composition | (μg/mL) | (μg/mL) | hour (μg/mL) |
| DMEM/F12 + 20% FBS | 0.040 | 1.090 | 10.9 |
| DMEM/F12 GlutaMAX supplemented + 20% FBS | 0.049 | 0.923 | 9.23 |
| DMEM/F12 + 20% FBS + 25 mM HEPES | 0.028 | 0.129 | 1.29 |
| DMEM/F12 + 20% FBS + 0.1% Pluronic F68 | 0.007 | 0.555 | 5.55 |

### Droplet Size

Table 5 shows droplet diameter changes after two rounds of picoinjection (virus followed by host cells).

TABLE 5
Droplet diameter change

| | | Estimated | Estimated |
|---|---|---|---|
| | Droplet | droplet | volume |
| | diameter (μm) | volume (pL) | change (%) |
| Original droplet size | 70.62 ± 0.36 | 184.4 | — |
| After 1^{st} picoinjection | 81.00 ± 0.92 | 278.3 | +50.9 |
| After 2^{nd} picoinjection | 89.70 ± 1.12 | 377.9 | +35.8 |

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A microfluidic method of assaying antibody secreting cells (ASCs), comprising the steps of:
a) isolating ASCs within droplets such that each droplet encapsulates only one ASC;
b) incubating the droplets of step a) to accumulate antibodies within the droplets;
c) picoinjecting virus into the droplets of step b) to form immune complex droplets;
d) picoinjecting host cells into the immune complex droplets to form neutralised droplets and infected droplets; and
e) sorting the infected droplets from the neutralised droplets, based on infection of the host cells by the virus, to assay the ASCs within the neutralised droplets.

2. The microfluidic method according to claim 1, wherein the neutralised droplets are sorted from the infected droplets using a dielectrophoretic sorter.

3. The microfluidic method according to claim 1, wherein the method further comprises a step before step a) of generating droplets in the presence of a lipopolysaccharide.

4. The microfluidic method according to claim 1, wherein the method further comprises a step after step c) of incubating the immune complex droplets and/or a step after step d) of incubating the neutralised droplets and infected droplets.

5. The microfluidic method according to claim 1, wherein the droplets of step a) and b) are characterised by one or both of:
a volume of about 160 pl to about 200 pl; and
a diameter of about 40 um to about 100 um, or preferably about 70 um to about 90 um.

6-8. (canceled)

9. The microfluidic method according to claim 1, wherein the method is characterised by a rate of about 200 droplets per second to about 500 droplets per second, or preferably about 300 droplets per second.

10. The microfluidic method according to claim 1, wherein the method further comprises a step of recovering the ASCs within the neutralised droplets; wherein the recovery step comprises demulsifying the neutralised droplets; and wherein the recovery step is characterised by an ASC enrichment ratio of more than about 1.8.

11. (canceled)

12. A microfluidic platform, comprising:
a) a droplet generator for generating droplets, each droplet encapsulating one antibody secreting cell (ASC), the droplet generator comprises an aqueous channel with an outlet for transporting the droplets and 2 oil channels intersecting the aqueous channel, the 2 oil channels configured to flow oil for pinching an aqueous medium in the aqueous channel into droplets;
b) a first picoinjector chip fluidly connected to the droplet generator, the first picoinjector chip comprising a first nozzle for delivering virus into the droplets to form immune complex droplets;
c) a second picoinjector chip fluidly connected to the first picoinjector chip, the second picoinjector chip comprising a second nozzle for delivering host cells into the immune complex droplets to form neutralised droplets and infected droplets; and
d) a droplet sorter fluidly connected to the second picoinjector chip, for sorting droplets based on infection of the host cells by the virus, the droplet sorter comprising a first channel and a second channel, the second channel configured to have a flow resistance greater than the first channel.

13. A microfluidic platform according to claim 12, wherein the second channel is configured to have a flow resistance at least 2 times that of the first channel; and/or wherein the droplet sorter comprises a channel having a width of more than about 100 μm.

14-15. (canceled)

16. The microfluidic platform according to claim 12, wherein the microfluidic platform further comprises a first vessel fluidly connected to the droplet generator, the first vessel configured to incubate the droplets;
a second vessel fluidly connected to the first picoinjector, the second vessel configured to incubate the immune complex droplets; and/or
a third vessel fluidly connected to the second picoinjector, the third vessel configured to incubate the neutralised droplets and infected droplets.

17-20. (canceled)

21. The microfluidic platform according to claim 12, wherein the first picoinjector contains fluid containing the virus, the fluid being at a pressure selected to deliver the virus to the droplets; and wherein the first picoinjector is configured with a first electric field, the first nozzle and the first electric field configured to act concurrently to deliver the virus to the droplets.

22. The microfluidic platform according to claim 12, wherein the second picoinjector contains fluid containing the host cells, the fluid being at a pressure selected to deliver the host cells to the immune complex droplets; and wherein the second picoinjector is configured with a second electric field, the second nozzle and the second electric field configured to act concurrently to deliver the host cells to the immune complex droplets.

23-25. (canceled)

26. The microfluidic method according to claim 1, wherein the droplets comprise:
a) a base medium comprising of Dulbecco's Modified Eagle Medium/Nutrient Mixture F-12 (DMEM/F-12), sodium bicarbonate at about 20 mg/L to about 30 mg/L, and sodium pyruvate at about 50 mg/L to about 60 mg/L;
b) fetal bovine serum at about 10% v/v to about 20% v/v of the base medium; and
c) a density gradient medium at about 10% v/v to about 20% v/v of the base medium.

27. The microfluidic method according to claim 1, wherein the droplets further comprise a mixture of penicillin G and streptomycin at about 1% v/v of the base medium.

28. The microfluidic method according to claim 1, wherein a volume of each of the neutralised droplets and infected droplets is about 65 pl to about 1000 pl and
wherein a volume of the neutralised droplets and infected droplets is less than 2 times a volume of the droplets; or
wherein the neutralised droplets and infected droplets are characterised by an about 30% to about 40% increase in volume relative to the immune complex droplets.

29. (canceled)

30. The microfluidic method according to claim 1, wherein the host cells are delivered at a cell density of about 80 million/mL to about 150 million/mL; and
wherein the neutralised droplets and infected droplets are characterised by a density of about 5 host cells per droplet to about 15 host cells per droplet.

31. The microfluidic method according to claim 1, wherein the ASCs are B cells or transfected cells, or preferably murine memory B cells.

32. The microfluidic method according to claim 1, wherein the droplets of step b) is characterised by an antibody concentration of about 0.1 μg/mL to about 20 μg/mL; and/or
wherein the immune complex droplets are characterised by a viral titer of about 5 kPFU/μL to about 100 kPFU/μL, or preferably about 75 kPFU/μL.

33. The microfluidic method according to claim 1, wherein the volume of each of the immune complex droplets is about 35 pl to about 800 pl; and wherein the immune complex droplets are characterised by an about 40% to about 60% increase in volume relative to the droplets.

34-41. (canceled)

42. The microfluidic method according to claim 1, wherein the virus is selected from Chikungunya virus, dengue virus, SARS-CoV-2, respiratory syncytial virus (RSV), Zika virus, EV71, influenza virus, HIV, and norovirus.
